(19) United States
(12) Patent Application Publication
(10) Pub. No.: US 2025/0360100 A1
(43) Pub. Date: Nov. 27, 2025

(54) ANTI-CANCER DERIVATIVES OF OVATODIOLIDE AND USE THEREOF

(71) Applicant: ARJIL BIOTECH HOLDING COMPANY LIMITED, Hsinchu City (TW)

(72) Inventors: Yeh B WU, Hsinchu City (TW); On LEE, Taipei City (TW); Hui Ju LIANG, Hsinchu City (TW); Wan Hsu YANG, Taipei City (TW)

(73) Assignee: ARJIL BIOTECH HOLDING COMPANY LIMITED, Hsinchu City (TW)

(21) Appl. No.: 19/210,944

(22) Filed: May 16, 2025

Related U.S. Application Data
(60) Provisional application No. 63/651,002, filed on May 23, 2024.

(51) Int. Cl. A61K 31/365; A61P 35/00; C07D 493/20; C07D 493/22

(52) U.S. Cl. CPC A61K 31/365; A61P 35/00; C07D 493/20; C07D 493/22

(57) ABSTRACT

The present invention pertains to a new derivative of ovatodiolide, which has unexpectedly good cytotoxicity in cancer cells. Also provided are the preparation process and the use of the new derivative of ovatodiolide.

### CROSS REFERENCE TO RELATED APPLICATION

This Non-provisional application claims the priority under 35 U.S.C. § 119 (e) on U.S. Patent Provisional Application No. 63/651,002 filed on May 23, 2024, the entire contents of which are hereby incorporated by reference.

### FIELD OF THE INVENTION

The present invention pertains to a new derivative of ovatodiolide, which has unexpectedly good cytotoxicity in cancer cells, and uses thereof.

### BACKGROUND OF THE INVENTION

Cancer is a type of diseases involving abnormal cell growth and has the potential of invading other organs or other parts of the body. Indications of cancer include a lump, abnormal bleeding, prolonged cough, unexplained weight loss, and a change in bowel movements. There were over 100 types of cancers that affect humans, while the most common types of cancer being lung cancer, prostate cancer, stomach cancer, breast cancer, colorectal cancer, and cervical cancer. For the past few years, cancer was diagnosed in 23.6 million people, and account for 10 million deaths worldwide annually.

Similar to cancer, tumors are another cause of death in human-beings, which occur as a result of accumulated genetic and epigenetic alterations within single cells and further cause the cell to divide and expand uncontrollably. However, in contrast to cancer, tumors do not spread between organs and body parts. The most common reason of the growth of a tumor is a neoplasm caused by an abnormal proliferation of tissues, which can be resulted from genetic mutation.

Ovatodiolide is isolated from Anisomeles indica (L) Kuntze (Labiatae), a traditional Chinese herb called ‘yu-chen-tsao’ in Chinese, which has been demonstrated to possess anti-inflammatory activity (Rao. Y. K. et al. The constituents of Anisomeles indica and their anti-inflammatory activities. J Ethnopharmacol 121, 292-296 (2009)). It has been reported that ovatodiolide exhibit many biological functions. For example, ovatodiolide is known as having anti-bacterial activities (Lien, H. M. et al. Bioevaluation of Anisomeles indica extracts and their inhibitory effects on Helicobacter pylori-mediated inflammation. J Ethnopharmacol 145, 397-401 (2013); Kulkarni, R. R. et al. Phyllocladane diterpenes from Anisomeles heyneana. J Asian Nat Prod Res 14, 1162-1168 (2012)) and anti-HIV activities (Shahidul Alam, M. et al. HIV-inhibitory diterpenoid from Anisomeles indica. Fitoterapia 71, 574-576 (2000)). In addition, ovatodiolide was ascertained have anti-cancer activities (Hou, Y. Y. et al. The natural diterpenoid ovatodiolide induces cell cycle arrest and apoptosis in human oral squamous cell carcinoma Ca9-22 cells. Life Sci 85. 26-32 (2009): Lin, K. L. et al. Antimetastatic effect and mechanism of ovatodiolide in MDA-MB-231 human breast cancer cells. Chem Biol Interact 194. 148-158 (2011); Ho. J. Y. et al. Ovatodiolide Targets beta-Catenin Signaling in Suppressing Tumorigenesis and Overcoming Drug Resistance in Renal Cell Carcinoma. Evid Based Complement Alternat Med 2013, 161628 (2013)). However, due to the poor solubility of ovatodiolide, the oral dosage form has disadvantages such as poor absorption, leading to reduced drug efficacy and difficulties in drug development.

It is desirable to develop some new derivatives having better properties and functions, particularly which can be used to develop a new anti-cancer drug.

### SUMMARY OF THE INVENTION

Accordingly, the present invention provides a new derivative of ovatodiolide exhibiting remarkable cytotoxic effects in cancer/tumor cells, and better solubility.

In one aspect, the present invention provides a new compound Ova-Oxy, which is a derivative of ovatodiolide, having one of the chemical structures below:

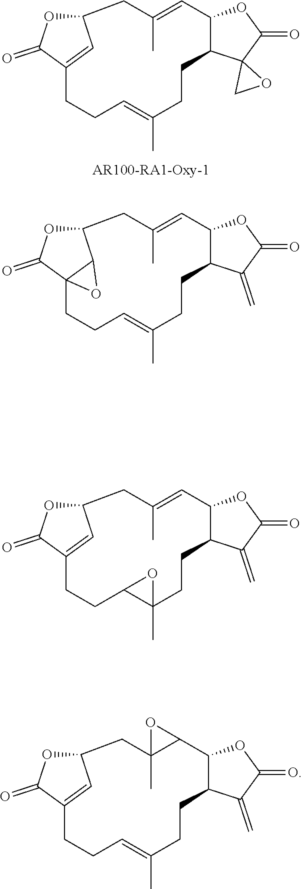

In the invention, the above-mentioned structures of the compound Ova-Oxy were identified by 1H NMR, LC-MS and the HPLC fingerprint of the compound Ova-Oxy were revealed.

In one example of the present invention, the compound Ova-Oxy has the structure below:

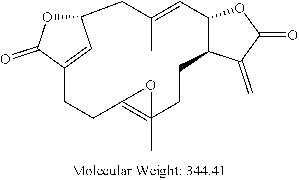

In another aspect, the present invention provides a process for preparation of the compound Ova-oxy, comprising an oxidation of ovatodiolide with an oxidative agent.

In one example, the oxidative agent is H₂O₂ and methyltrioxorhenium (VII) (MTO).
(a) adding ovatodiolides in dichloromethane (DCM), H₂O, H₂O₂ and methyltrioxorhenium (VII) (MTO) at about 0° C. to obtain a solution;
(b) stirring the solution at room temperature overnight;
(c) extracting an aqueous layer of the solution with dichloromethane (DCM) to collect the organic layers to obtain a combined organic layer;
(d) drying the combined organic layers over Na₂SO_{4,} and filtering and concentrating the combined organic layers under a reduced pressure, purifying the resultant with silica gel column chromatography eluting with DCM to provide the compound Ova-Oxy as white solid.

In a further aspect, the present invention provides a pharmaceutical composition comprising the compound Ova-Oxy and a pharmaceutically acceptable carrier.

In one embodiment of the present invention, the pharmaceutical composition is effective for treating and/or preventing a cancer or a tumor in a subject.

In a yet aspect, the invention, the present invention also provides the use of the compound Ova-Oxy for manufacturing a medicament for treating and/or preventing a cancer or a tumor.

As an alternative, the present invention provides a method for treating or preventing a cancer or a tumor in a subject comprising administering said subject a therapeutically effective amount of the compound Ova-Oxy or a pharmaceutical composition thereof.

In the examples of the invention, the Ova-Oxy compound is ascertained to exhibit cytotoxic effects in all of the seven cancer cell lines, indicating that the compound Ova-Oxy is potential as a therapeutic agent for treating a cancer or a tumor, wherein the cancer comprises kidney cancer, lung carcinoma, colorectal cancer, liver cancer, prostate cancer, pancreas cancer, or gastric adenocarcinoma.

In a further yet aspect, the present invention provides a pharmaceutical composition for treating a cancer comprising a therapeutically effective amount of the compound Ova-Oxy and a pharmaceutically acceptable carrier.

In one embodiment of the invention, the pharmaceutical composition for treating a cancer further comprises an additional therapeutic agent.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred.

In the drawings:

FIG. 1 shows the HPLC (High Performance Liquid Chromatography) fingerprint of the compound Ova-Oxy.

FIG. 2 shows the LC-MS (Liquid Chromatography Mass Spectrometry) fingerprint of the compound Ova-Oxy.

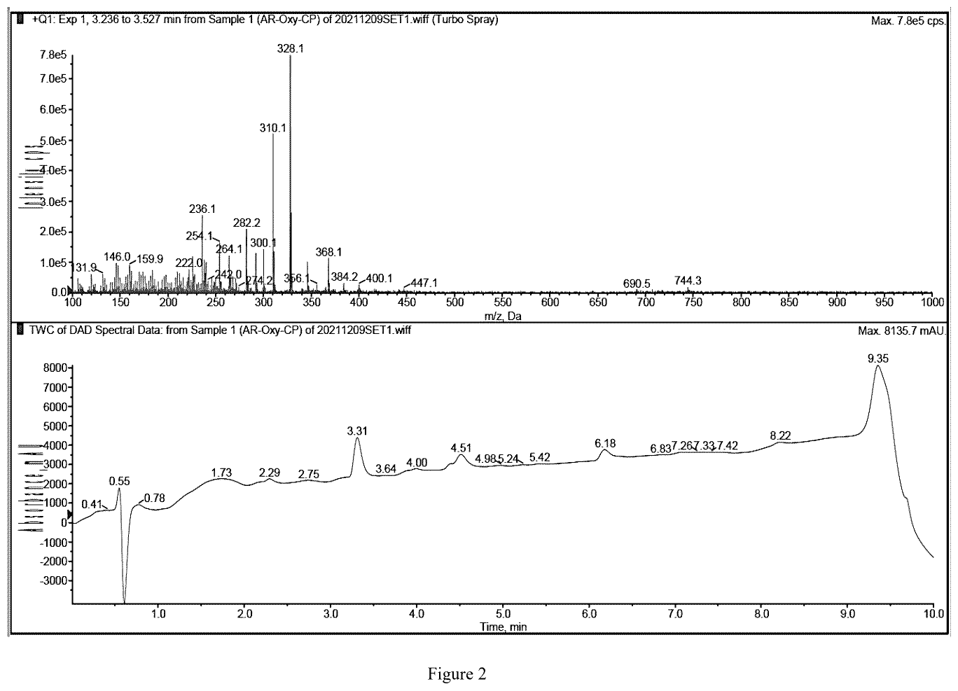

FIG. 3 shows the NMR (Nuclear Magnetic Resonance) analysis result of the compound Ova-Oxy.

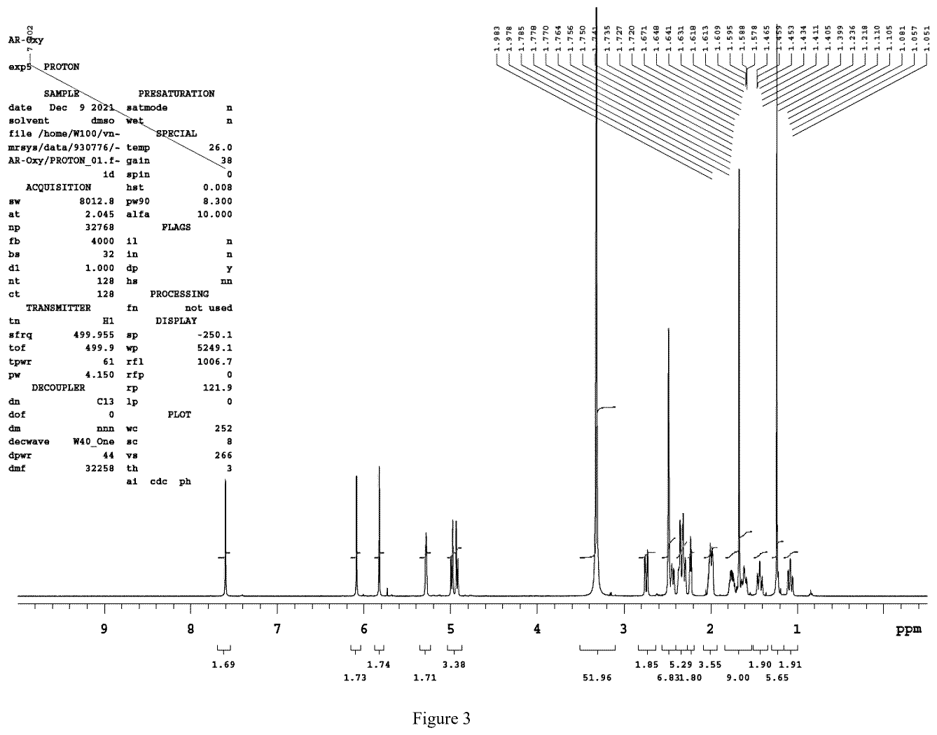

### DETAILED DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a sample” includes a plurality of such samples and equivalents thereto known to those skilled in the art.

As used herein, the phrase “therapeutically effective amount” refers to an amount sufficient to treat a disease at a reasonable benefit/risk ratio applicable to medical treatment, and an effective dose level may be determined by the species and severity, age, sex, type of disease, duration of treatment, factors including co-administered drugs, and factors well known in other medical disciplines.

As used herein, the term “subject” refers to a human or a mammal, preferably a human. The term “individual”, “subject”, and “patient” are used interchangeably.

As used herein, the term “therapeutically effective amount” refers to the amount that is safe and sufficient to prevent or delay the development and further spread of metastases in cancer patients. The amount can also cure or cause the cancer to go into remission, slow the course of cancer progression, slow or inhibit tumor growth, slow or inhibit tumor metastasis, slow or inhibit the establishment of secondary tumors at metastatic sites, or inhibit the formation of new tumor/cancer metastasis.

As used herein, the term “treat” or “treatment” refers to both therapeutic treatment and prophylactic or preventative measures, wherein the object is to prevent, slow down, and/or halt the development or spread of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already diagnosed with cancer as well as those likely to develop secondary tumors due to metastasis.

As used herein, the terms “pharmaceutically acceptable carrier” or “pharmaceutically acceptable excipient” is intended to include any and all solvents, dispersion media, coatings, isotonic and absorption delaying agents, and inert ingredients. The use of such pharmaceutically acceptable carriers or pharmaceutically acceptable excipients for active pharmaceutical ingredients is well known in the art.

As used herein, the term “ovatodiolide,” also called as “AR100-RA1” or “Ova,” refers to a compound having a chemical structure as shown below:

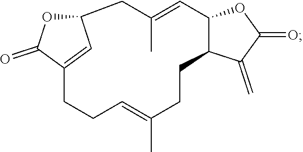 [SMILES: C=C1C(=O)O[C@H]2/C=C(\C)C[C@@H]3C=C(CC/C=C(\C)CC[C@H]12)C(=O)O3]

which is a candidate compound of Arjil pharmaceuticals Co., Ltd., showing extraordinary effects on treating cardiovascular disease and alleviating cytotoxicity.

However, ovatodiolide is of poor solubility, and its oral dosage form has disadvantages such as poor absorption, leading to reduced drug efficacy. To solve the problem of ovatodiolide, the present prevention provides a derivative to enhance the solubility.

The present invention provides a derivative of Ovatodiolide, called as a compound “Ova-Oxy” or “AR100-RA1-Oxy,” as represented by one of the four structures below:

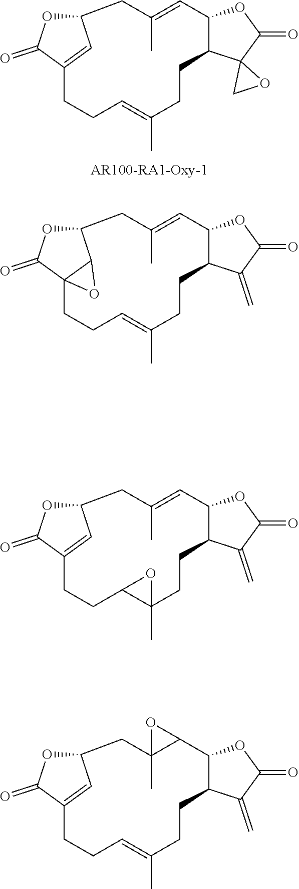

In a particular example of the present invention, the compound Ova-Oxy has the structure below:

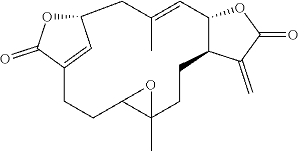 [SMILES: C=C1C(=O)O[C@H]2/C=C(\C)C[C@@H]3C=C(CCC4OC4(C)CC[C@H]12)C(=O)O3]

According to the invention, the compound Ova-Oxy is prepared by an oxidation of ovatodiolide, as shown in the scheme below:

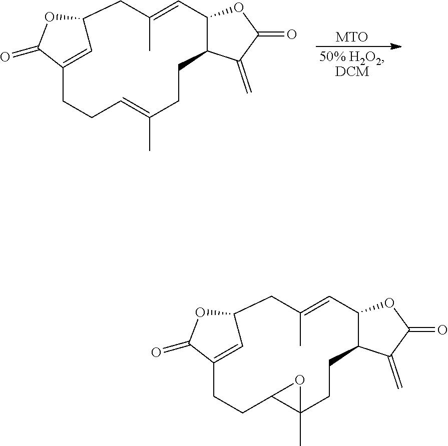

In a certain embodiment, the compound “Ova-Oxy” is synthesized by a process having the step of adding ovatodiolides in dichloromethane (DCM), H₂O, H₂O₂ and methyltrioxorhenium (VII) (MTO) to obtain a solution and keeping the solution at about 0° C., stirring the solution at room temperature, extracting an aqueous layer with DCM, drying combined organic layers over Na₂SO₄, filtering and concentrating the organic layers under reduced pressure, and purifying the resultant with silica gel column chromatography eluting with DCM to provide the compound Ova-Oxy as a white solid.

In the invention, the cytotoxic effect was evaluated by the viability of cancer/tumor cells measured by the MTT assay, in several cancer cell lines, including the cell lines 293, A549, Colo205, SK-HEP1, PC-3, PANC-1, and AGS.

In the invention, the Ova-oxy are synthesized, and confirmed to exhibit unexpectedly better properties and medical effects. It is demonstrated that the compound Ova-Oxy exhibits a remarkable cytotoxic effect on tumor/cancers, including but not limited to the cell lines 293, A549, Colo205, SK-HEP1, PC-3, PANC-1, and AGS.

In the invention, the compound Ova-Oxy can be formulated or prepared as a pharmaceutical composition. The pharmaceutical composition comprises, for example, the compound Ova-Oxy and a pharmaceutically acceptable carrier.

As used herein, the phrase “pharmaceutically acceptable carrier” may encompass a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. The pharmaceutically acceptable carriers that are well known in the art can be used in the present invention.

The pharmaceutical composition can be administered to a subject by any of a number of routes of administration including, for example, intraocularly (for example, by intraocular injection); and topically (for example, as a cream, ointment or spray applied to the skin, or as an eye drop). In certain embodiments, a compound may be simply dissolved or suspended in sterile water.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both.

For use in the methods of this invention, the compound Ova-Oxy can be given per se or as a pharmaceutical composition containing, for example, 0.1 to 99.5% (more preferably, 0.5 to 90%) of active ingredient in combination with a pharmaceutically acceptable carrier.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

The present invention is further illustrated by the following examples, which are provided for the purpose of demonstration rather than limitation.

### EXAMPLES

### Example 1

### Preparation of the Compound Ova-Oxy

Ovatodiolide (4.50 g, 13.7 mmol, 1.0 eq) in DCM (50 mL) was added with H₂O (20.0 mL), 50% H₂O (3.0 mL, 41.1 mmol, 3.0 eq), methyltrioxorhenium (VII) (MTO) (135.0 mg, 3%) at 0° C., and stirred at room temperature overnight. MTO was used as an oxidative agent in the synthesis of the compound Ova-Oxy. The layers were later separated and the aqueous layer was extracted with DCM (2×100 mL), while the combined organic layers were dried over Na₂SO₄, filtered and concentrated under reduces pressure. The resultant was purified by silica gel column chromatography eluting with DCM to provide the compound Ova-Oxy (2.04 g, yield 43.2%) as white solid.

### Example 2

### Structural Analysis of the Compound Ova-Oxy

The structure of the compound Ova-Oxy has been further verified by 1H NMR and LC-MS under the conditions as shown in Table 1 below.

TABLE 1
The conditions for the analysis of Ova-Oxy by HPLC.

| | | | | |
|---|---|---|---|---|
| Column | Inertsil ODS-3, 5 μm, | UV | 210 | nm |
| | 4.6 × 250 mm | | | |
| Mobile phase | 0.1% TFA in 30% CAN | Running Time | 30.0 | min |
| Flow rate | 1.0 mL/min | Injection Vol | 20 | Ml |

| | |
|---|---|
| Sample RT. | 21.59 |
| (min) | |

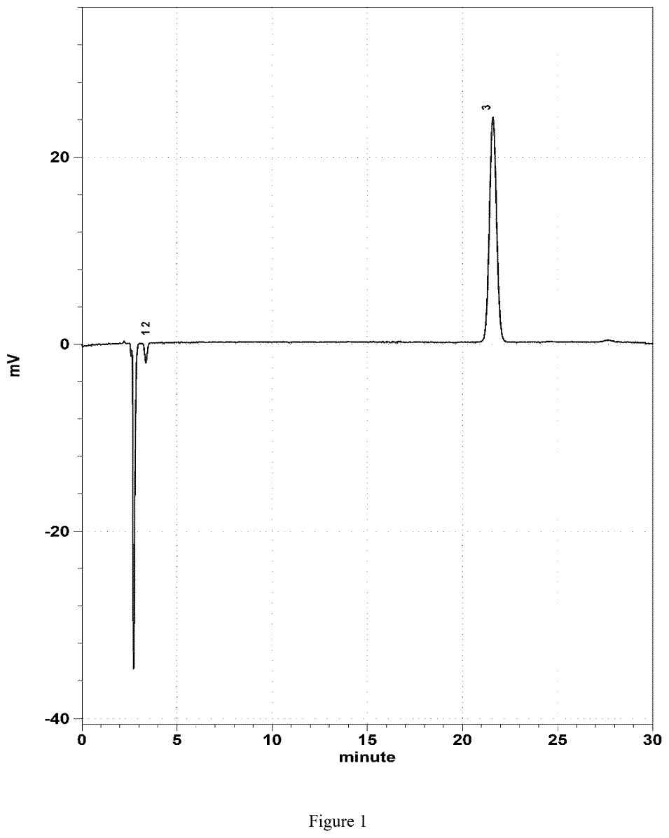

The results are shown in FIG. 1 and Table 2 below. In the synthesis of the compound Ova-Oxy, the structures have been successfully confirmed by ¹H NMR and LC-MS.

TABLE 2
Peak information from the HPLC analysis result of OVA-oxy.

| Peak | Retention | Area | | | |
|---|---|---|---|---|---|
| No. | Time | (uv*sec) | Area % | Height | Height % |
| 1 | 3.038 | 248421 | 28.3932 | 5.7196 | 19.1968 |
| 2 | 3.676 | 12320 | 1.4081 | 0.2010 | 0.6747 |
| 3 | 21.596 | 614191 | 70.1988 | 23.8742 | 80.1285 |
| Total | | 874932 | | 29.7948 | |

### Example 3

### Effects of the Compound Ova-Oxy on Cancer Cell Lines

The cytotoxicity of the compound Ova-Oxy according to the invention, ovatodiolide (AR100-DS1) and a reference compound Ova-ST2 as the compounds as tested, provided by Arjil Pharmaceuticals LLC were evaluated. The compound Ova-ST2 has the chemical structure:

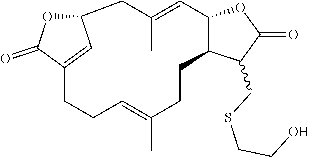

The compounds as tested were dissolved in DMSO (100 mg/mL), along with Doxorubicin (Dox), taken as a benchmark control, and the in vitro cytotoxicity in cancer cell lines were tested from 60 or 100 μg/mL with three-fold serial dilutions, and the 50% cytotoxic concentration (CC50) of test articles was determined by MTT assay.

Seven cancer cell lines were used in this example, including 293 (human embryonic kidney cells 293), A549 (human non-small cell lung cancer cells), Colo205 (human colon adenocarcinoma cells), SK-HEP1 (human hepatic cancer cells), PC-3 (human prostate carcinoma), PANC-1 (human pancreatic epithelioid carcinoma), and AGS (human gastric adenocarcinoma cells), cultured in the media and indicated as the table above. Each of the cell lines and its cell number and culturing medium are given in Table 3. Briefly, cells seeded in 96-well plates were treated for 48 hours with the compounds as tested and the solvent (0.1% DMSO) as the vehicle control, respectively. Cell viability was determined by the MTT method, and the CC50 values were calculated using GraFit (Version 7, Erithacus Software Ltd., Horley, UK) and recorded.

TABLE 3
The cell lines and cell numbers and culturing media thereof

| Cell | Cell | |
|---|---|---|
| line | Number | Medium |
| 293 | 1 × 10⁵ | Eagle's minimum essential medium, 10% fetal |
| | cells/ml | bovine serum, 2 mM L-glutamine, 1.5 g/L sodium |
| | | bicarbonate, 0.1 mM non-essential amino acids, |
| | | and 1.0 mM sodium pyruvate. |
| A549 | 8 × 10⁴ | Ham's F12K medium, 10% fetal bovine serum, |
| | cells/ml | 2 mM L-glutamine, and 1.5 g/L sodium bicarbonate. |
| Colo205 | 2 × 10⁵ | RPMI 1640 medium, 2 mM L-glutamine, 1.5 g/L |
| | cells/ml | sodium bicarbonate, 4.5 g/L glucose, 10 mM |
| | | HEPES, and 1.0 mM sodium pyruvate. |
| SK-HEP1 | 3 × 10⁴ | Eagle's minimum essential medium, 10% fetal |
| | cells/ml | bovine serum, 2 mM L-glutamine, 1.5 g/L sodium |
| | | bicarbonate, 0.1 mM non-essential amino acids, |
| | | and 1.0 mM sodium pyruvate. |
| PC-3 | 5 × 10⁴ | Ham's F12K medium, 7% fetal bovine serum, |
| | cells/ml | 2 mM L-glutamine, and 1.5 g/L sodium |
| | | bicarbonate. |
| PANC-1 | 5 × 10⁴ | Dulbecco's modified Eagle's medium, 10% |
| | cells/ml | fetal bovine serum, 4 mM L-glutamine, 1.5 g/L |
| | | sodium bicarbonate, and 4.5 g/L glucose. |
| AGS | 1 × 10⁵ | Ham's F-12K medium and 10% fetal bovine |
| | cells/ml | serum. |

The CC50 values of the compounds as tested in the cell models of the 293, A549, Colo205, SK-HEP1, PC-3, PANC-1, and AGS cells, respectively are shown in Table 4. In view of the results shown in Table 4, it was indicated that the Ova-Oxy exhibited similar as an even better cytotoxicity than ovatodiolide (AR100-DS1); however, another derivative of Ova-ST2, did not exhibit the cytotoxicity. Particularly, it was unexpected that the compound Ova-Oxy has the low CC50 value (17.0±0.2 μg/mL) in the Colo205 cells.

TABLE 4
The CC50 values of the compound as tested in the cell models. (μg/mL)

| No. | Code | 293 | A549 | Colo205 | SK-HEP1 | PC-3 | PANC-1 | AGS |
|---|---|---|---|---|---|---|---|---|
| 1 | AR100-DS1 | 25.7 ± 2.5 | 29.6 ± 0.4 | 41.7 ± 4.5 | 49.9 ± 5.2 | 17.8 ± 0.2 | 40.2 ± 3.9 | 13.0 ± 5.1 |
| 2 | Ova-Oxy | 22.5 ± 0.5 | 23.0 ± 4.7 | 17.0 ± 0.2 | 45.2 ± 3.2 | 12.3 ± 5.4 | 32.0 ± 0.8 | 9.1 ± 0.9 |
| 3 | Ova-ST2 | >100 | >100 | >100 | >100 | >100 | >100 | >100 |
| 4 | Dox | >10 | >10 | 8.7 ± 0.6 | 9.8 ± 0.2 | 10.3 ± 1.5 | 10.7 ± 1.5 | 0.3 ± 0.1 |

In conclusion, the compound Ova-Oxy exhibits unexpectedly better cytotoxicity in cancer cells than ovatodiolide and other derivatives.

The present invention provides the method of treating cancers comprising kidney cancer, lung carcinoma, colorectal cancer, liver cancer, prostate cancer, pancreas cancer, and gastric adenocarcinoma by Ova-Oxy.

While this specification contains many specifics, these should not be construed as limitations on the scope of the invention or of what may be claimed, but rather as descriptions of features specific to particular embodiments or examples of the invention. Certain features that are described in this specification in the context of separate embodiments or examples can also be implemented in combination in a single embodiment.

What is claimed is:

1. A compound Ova-Oxy having a chemical structure selected from the four structures below:

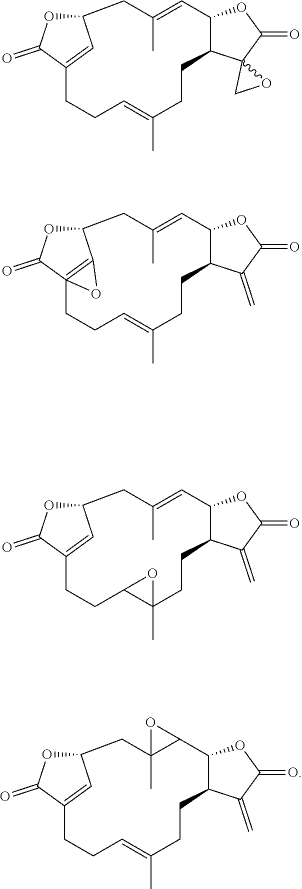

2. The compound of claim 1, which has the chemical structure of

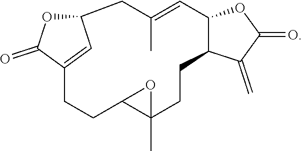 [SMILES: C=C1C(=O)O[C@H]2/C=C(\C)C[C@@H]3C=C(CCC4OC4(C)CC[C@H]12)C(=O)O3]

3. A process for preparing the compound according to claim 1, comprising an oxidation of ovatodiolide, which comprises the steps of:
(a) adding Ovatodiolides in dichloromethane (DCM), H₂O, H₂O₂ and methyltrioxorhenium (VII) (MTO) at 0° C. to obtain a solution;
(b) stirring the solution at room temperature overnight;
(c) extracting an aqueous layer of the solution with dichloromethane (DCM) to collect the organic layers to obtain a combined organic layer;
(d) drying the combined organic layers over Na₂SO₄, and filtering and concentrating the combined organic layers under a reduced pressure, purifying the resultant with silica gel column chromatography eluting with DCM to provide the compound Ova-Oxy as white solid.

4. A pharmaceutical composition comprising the compound Ova-Oxy set forth in claim 1 and a pharmaceutical acceptable carrier.

5. The pharmaceutical composition of claim 4, in which the compound Ova-Oxy has the chemical structure of

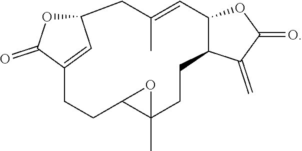 [SMILES: C=C1C(=O)O[C@H]2/C=C(\C)C[C@@H]3C=C(CCC4OC4(C)CC[C@H]12)C(=O)O3]

6. The pharmaceutical composition of claim 4, which is effective for treating or preventing a cancer or a tumor in a subject.

7. A method for treating or preventing a cancer or a tumor in a subject, comprising administering to said subject a therapeutically effective amount of the compound as defined in claim 1 or the pharmaceutical composition comprising a therapeutically effective amount of the compound according to claim 1, and a pharmaceutically acceptable carrier.

8. The method of claim 7, which further comprises an additional therapeutic agent.

9. The method of claim 7, wherein the cancer comprises kidney cancer, lung carcinoma, colorectal cancer, liver cancer, prostate cancer, pancreas cancer, or gastric adenocarcinoma.
